(19) United States
(12) Patent Application Publication
(10) Pub. No.: US 2026/0307174 A1
(43) Pub. Date: Oct. 8, 2026

(54) METHOD OF MAKING LARGESURFACE-AREA MICROFLUIDIC DEVICES AND MICROFLUIDIC LUNG MANUFACTURED USING SAID METHOD

(71) Applicants: The United States Government as represented by the Department of Veterans Affairs, Washington, DC (US); The Regents of the University of Michigan, Ann Arbor, MI (US)

(72) Inventors: Joseph A. Potkay, Ann Arbor, MI (US); Andrew Zhang, Ann Arbor, MI (US)

(21) Appl. No.: 19/491,447

(22) PCT Filed: Jun. 13, 2024
(86) PCT No.: PCT/US2024/033754

§ 371 Date: Dec. 9, 2025

Related U.S. Application Data
(60) Provisional application No. 63/507,891, filed on Jun. 13, 2023.

(51) Int. Cl. B23K 26/364; A61M 1/16; B23K 26/402; B23K 103/00; B65H 39/00; B65H 41/00

(52) U.S. Cl. CPC B23K 26/364; A61M 1/1698; B23K 26/402; B65H 39/00; B65H 41/00; A61M 2207/10; B23K 2103/42; B65H 2301/4695

(57) ABSTRACT

Systems and methods for forming a microfluidic device are disclosed. Microfluidic channels are formed into a substrate. The substrate can be formed into a roll to define a plurality of layers and bonded to itself. The substrate can be at least partly positioned within a housing.

### CROSS-REFERENCE TO RELATED APPLICATION

This application claims priority to and the benefit of the filing date of U.S. Provisional Patent Application No. 63/507,891, filed Jun. 13, 2023, the entirety of which is hereby incorporated by reference herein.

### FIELD

This disclosure relates to gas exchange devices such as a microfluidic artificial lung and systems and methods for making such gas exchange devices.

### BACKGROUND

Gas exchange can be beneficial for various applications, including microfluidic artificial lungs. Most microfluidic artificial lungs have only a single layer which limits their rated flows. Efforts to scale up these devices, such as by stacking multiple flat microfluidic artificial lungs have been labor intensive and resulted in bulky devices. Accordingly, less bulky and less labor-intensive devices are desirable.

### SUMMARY

Disclosed herein, in various aspects, is a roll-to-roll system for forming a microfluidic device. The microfluidic device can be a multilayer microfluidic device.

Also disclosed herein, in various aspects, are methods of forming such microfluidic devices.

Also disclosed herein, in various aspects, are microfluidic devices.

In one aspect, a system includes a laser engraver configured to form a plurality of channels into a substrate, the substrate having a first surface and a second surface opposite the first surface. A take-up roll is configured to roll the substrate with the plurality of channels formed therein. A bonder is configured to bond a portion of the first surface of the substrate to an adjacent portion of the second surface of the substrate.

In one aspect, a method of making a microfluidic gas exchanger includes engraving a plurality of channels into a substrate; winding the substrate onto a take-up roll; and bonding a first surface of a first portion of the substrate to a second surface of a second portion of the substrate that is adjacent the first portion along a radial axis.

A microfluidic lung includes a substrate wound into a roll. The substrate comprises a plurality of channels formed therein. The substrate has a first surface and a second surface opposite the first surface. At least a portion of the first surface of the substrate is bonded to an adjacent portion of the second surface of the substrate.

### BRIEF DESCRIPTION OF THE DRAWINGS

FIG. 1 is a schematic diagram of a system of forming a multi-layer microfluidic device as disclosed herein.

FIG. 2 shows a confocal scan of engraved Silpuran 2030. Channels are 60 μm deep and 180 μm wide.

FIG. 3 illustrates a custom housing of a microfluidic device.

FIG. 4 shows a microfluidic device with colored water being routed through. Red arrows depict blood connections, and blue arrows depict gas connections. Two of the blood outlets are filled with blue dye, and there is a dark blue region indicating imperfect layer-to-layer bonding resulting in leaking.

FIG. 5 is a graph showing experimental vs theoretical pressure drop across the μAL (water).

FIG. 6 is a schematic diagram of a substrate formed into a roll.

FIG. 7 is a schematic diagram of the substrate of FIG. 6, with a segment un-rolled to illustrate channels therein.

FIG. 8 illustrates a portion of an exemplary system, showing a laser engraver (B) engraves channels into PDMS, Cold plasma wands (C) allow PDMS layers to adhere to each other, ending on finished cylindrical PDMS roll (D), with waste on a rewinder (E).

FIG. 9A shows a cross-section with radially fixed positioning of each layer. There are clear regions where each fluid can be accessed in bulk. FIG. 9B shows a cross-section with linearly fixed engraving length and spacing. Because the diameter changes as more layers are added, the positioning appears random. There is no way to access a particular feature without unrolling the processed material.

FIG. 10 shows an adjustable laser engraving vacuum table.

FIG. 11A shows plasma-activating the outer, non-engraved side of the PDMS film after it is incorporated into the rewinder, which allows the length of PDMS film without the PETG backing is minimized. FIG. 11B shows a method which separates plasma integration and rewinding into two separate steps involves plasma-activating both sides of the PDMS at once. There is a long distance where the PDMS film is without its PETG backing before it reaches the rewinder. This causes difficulty in the rerolling process due to stretching and wrinkling of the film. (FIGS. 11A and 11B not shown to scale.)

FIG. 12 shows a gas layer and blood layer overlaid on each other. Vertical lines denote blood channel engravings, and horizontal lines denote gas channel engravings. The dotted horizontal lines denote where the core is cut after completion of the R2R process to expose the blood channels, and the dotted vertical lines denote where the completed core is cut to expose the gas channels. (Not shown to scale.)

FIG. 13 shows a drawing of a device showing alternating blood and gas layers. Since each blood layer has a gas layer both above and below it, gas can diffuse in two directions, greatly increasing the gas exchange capability. Blood travels axially, while gas travels circumferentially. (Not shown to scale.)

FIG. 14A shows an exemplary fluidics device. Vertical lines show where the core is cut to expose the blood channels. The horizontal line shows one location where the core is cut to expose the gas channels, with the second being on the other side. FIG. 14B shows a cutting rig for cutting the processed output roll to the desired width. FIG. 14C shows a cut output roll. On the inside surface of the plastic inner core, teeth can be seen that provide friction to keep the side and main pieces together during processing.

FIG. 15A shows a computer model of the device, labeled with relevant fluidic features. FIG. 15B shows a computational fluid simulation was used to confirm relative flow velocities and distribution.

FIG. 16 shows the uAL-testing setup used to test the devices.

FIG. 17 shows device C filled with food coloring for visualization, labeled, to show the major features.

FIG. 18A is a plot illustrating a pressure drop of each device with water, and Device B with blood. FIG. 18B: is a plot illustrating resistance as a function of flow rate.

FIG. 19A illustrates a water testing with Device A. Gas flow was 0.025 mL/min. This is a simplified test which only tested CO₂ addition. The pH sensor was not calibrated, the water was not conditioned beforehand to remove existing CO₂, and CO₂ removal was not tested for this device. Nevertheless, the sharp decrease in pH indicates the addition of CO₂.

FIG. 19B ‡ Water testing with Device B. The area between the two orange lines represents when the CO₂ is set to 100% for 3 hours, and a corresponding drop and then rise in pH can be seen. Gas flow was set to 0.5 mL/min. The full 46-hour test including calibration and conditioning is shown in Supplemental Information, Figure S3.

FIG. 20 shows a plot of outlet blood Saturation as a function of Flow Rate during blood testing for Device B. Error bars represent 95% confidence intervals, based on the standard deviation from the last 30s of O₂ saturation readings.

FIG. 21 shows a confocal image of Device B cut open. 7 layers of blood channels can be clearly observed, along with the other layers of the device.

FIG. 22 shows a plot of gas exchange by flow rate, calculated using saturation values from FIG. 20. Error bars represent 95% confidence intervals, based on the standard deviation from the last 30s of O₂ saturation readings.

FIG. 23 shows a cutting rig which is used to cut the PDMS to exactly 60 mm width.

FIG. 24 is a housing CAD model for Device B and C.

FIG. 25 illustrates a housing and Caulking Technique for Device B and C, blood phase. Caulking is applied to the corners denoted in cyan. Capillaries (red) mostly feed into the empty chamber in the housing. The area denoted in blue represents the area where the core comes in contact with the housing. Unlike the caulking technique used for Device A, there is no silicone caulking in this area. Since caulking does not spill into the flowpath, the number of capillaries available is not a function of the caulking, but only by the number of patent capillaries facing the blood distribution area in the housing. For Device A, the gaps where silicone caulking could be applied to the points in cyan did not exist. Therefore caulking was applied to the equivalent location in blue. The caulking would spill out into the blood distribution, blocking many of the capillaries.

FIG. 26 illustrates a housing and Caulking Technique for Device B and C, gas phase, and scheduling of assembly. Day 1: The caulking for the blood phase (FIG. 25) is applied and allowed to cure. Day 2: The lower half of the gas manifold is installed. Silicone caulking is applied at the locations denoted in purple and allowed to cure. This serves the dual purpose of keeping the gas phase sealed and preventing the device from coming apart in the next step. Day 3: A razor is used to cut downwards into the core (red) and through the gas capillaries which are traveling which run circumferentially around the diameter of the device and are passing perpendicularly to the plane of this incision. The tension of the PDMS pulls the core open where the incision is made, exposing these gas capillaries. Since the caulking has been given time to cure, this opening does not spread beyond the area encircled by the caulking (purple). Silicone caulking is applied to the area where the lower and upper halves of the gas manifold meet (green), and the top half of the gas manifold is installed.

FIG. 27 shows a plot illustrating the full length of the water pH testing. The system is given 24 h to stabilize. The area between the two orange lines represents when the CO₂ is set to 100% for 3 hours, and a corresponding drop and then rise in pH can be seen.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” can optionally include plural references unless the context clearly dictates otherwise. Thus, for example, reference to “a layer” can represent disclosure of embodiments including only a single such layer, as well as embodiments including a plurality of such layers, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

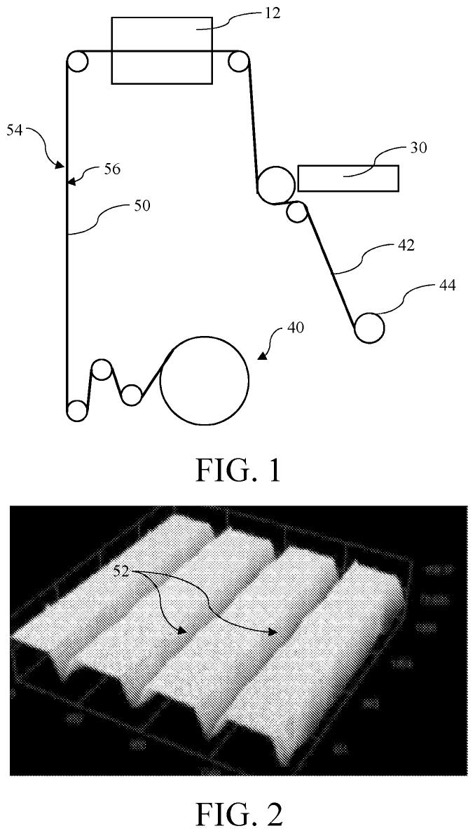

Disclosed herein, in various aspects, and with reference to FIGS. 1-7, are roll-to-roll systems for forming microfluidic devices (e.g., a multi-layer microfluidic device), methods of forming and/or using such microfluidic devices, and the microfluidic devices produced or used by such systems and methods.

Referring to FIG. 1, a system 10 for forming a multi-layer microfluidic device can comprise a channel forming device 12 for forming a plurality of channels 52 (FIG. 2) in a substrate 50. A take-up roll 20 can be configured to roll the substrate with the plurality of channels formed therein. A bonder 30 (e.g., one or more cold plasma wands) can be configured to bond a portion of the first surface 54 of the substrate 50 to an adjacent portion of the second surface 56 of the substrate.

In exemplary aspects, the channel forming device 12 can be a laser engraver. In additional aspects, the channel forming device 12 can be an embosser that is configured to emboss channels into the substrate. In further exemplary aspects, the channel forming device 12 can comprise one or a plurality of knives that are configured to cut the channels into the substrate 50. In still further aspects, the channel forming device 12 can comprise a mold that permits inline molding of the substrate 50 with the plurality of channels 52 formed therein.

In some aspects, the bonder 30 can comprise a plurality of (e.g., a pair of) of cold-plasma wands. The cold-plasma wands can be spaced along a rotational axis 22 of the take-up roll 20. In additional aspects, the bonder 30 can comprise one or more of: an oxygen plasma source, an ozone source for exposing the substrate to ozone, a device for providing corona discharge treatment, or an acid source for providing acid exposure. Optionally acid exposure can comprise exposure to gaseous acid. Optionally, the acid can comprise hydrochloric acid (e.g., gaseous hydrochloric acid). More generally, the bonder 30 can be configured to activate a surface of the substrate to create silanol groups.

The substrate 50 can be fed to the channel forming device 12 from a supply roll 40. Optionally, the supply roll can comprise, for example, the substrate 50 and a backing material 42. In some aspects, the system 10 can comprise a second take-up roll 44 configured to wind the backing layer 42 therearound to draw the backing layer from the substrate 50. It is contemplated that the backing layer 42 can have a fixed length, whereas the substrate can be subject to stretching. Accordingly, the second take-up roll 44 can be driven by a motor to control movement of the substrate 50 through the system 10. The take-up roll 20 can be controlled to rotate relative to the speed of the second take-up roll 44, accounting for the diameter of the substrate 50 wound around the take-up roll 20 and the diameter of the backing material 42 wound around the second take-up roll 44 to control tension of the substrate as it is wound around the take-up roll 20.

Optionally, the substrate 50 can comprise a removable cover layer on a surface into which the channel forming device 12 forms the channels 52. In this way, the cover layer can protect the surface of the substrate 50 during formation of the channels (e.g., to protect the surface from debris, thereby allowing for better bonding). Optionally, the backing layer 42 can serve as said cover layer. In other aspects, the backing layer 42 can be provided on a first side of the substrate 50, and the cover layer can be provided on a side of the substrate opposite the backing layer 42.

In some aspects, the substrate 50 can comprise silicone. For example, the substrate 50 can comprise, consist of, or consist essentially of, polydimethylsiloxane (PDMS). In additional aspects, the substrate can comprise a reverse-selective polymer. For example, the substrate 50 can comprise poly tert-butylacetylene (PTBA), poly 4-methyl-2-pentyne (PMP), or poly 1-trimethylsilyl-1-propyne (PTMSP). In some aspects, the substrate 50 can be gas permeable. In this way, the substrate 50 can be configured to permit gas exchange therethrough. This can be advantageous for forming an artificial lung. Optionally, the substrate 50 can be porous. For example, the porous substrate 50 can comprise porous polypropylene or porous polymethylpentene. It is contemplated that porosity can provide selectable gas permeability. Optionally, the gas permeability can be relatively high as compared to other nonporous substrates). In other aspects, the substrate 50 can be gas impermeable or substantially gas impermeable.

A method of making a microfluidic gas exchanger can comprise engraving a plurality of channels 52 into a substrate 50. The substrate 50 can then be wound onto a take-up roll 20.

Referring to FIG. 7, in exemplary aspects, the plurality channels 52 can be formed so that, when the substrate 50 is wound onto the take-up roll 20, channels of adjacent layers 70 can be configured to carry different fluids. For example, a first layer 70a can define a first plurality of channels 52a configured to carry a first fluid (e.g., gas), and a second layer can define a second plurality of channels 52b configured to carry a second fluid (e.g., blood).

The substrate 50 can be gas-permeable so that gas can exchange between the adjacent channels of the first and second layers. In still further aspects, the microfluidic device can be formed so that the channels of the first and second layers are fluidly isolated from each other, except for gas exchange through the substrate 50.

In some aspects, the first and second pluralities of channels can extend transversely to (optionally, orthogonal to) each other. For example, the first plurality of channels 52a can extend circumferentially, and the second plurality of channels 52b can extend axially. Accordingly, in one embodiment, gas can flow through first plurality of channels, and blood can flow axially through second plurality of channels. In some aspects, gas can be transferred both radially inwardly and radially outwardly between adjacent channels through the substrate 50.

In further aspects, when the substrate 50 is wound onto the take-up roll 20, layers 70 can alternatingly define the first and second pluralities of channels. In these aspects, the first plurality of channels can be fluidly isolated from the second plurality of channels, except for gas exchange through the substrate 50. For example, the substrate 50 can comprise between 10 and 1000 layers of alternating first and second pluralities of channels 52a,b. For example, the substrate 50 can be wound to have at least 100 layers of alternating first and second pluralities of channels 52a.b. In additional aspects, when the substrate 50 is wound onto the take-up roll 20, the roll can have from about 80 layers to about 800 layers, or about 100 layers to about 500 layers.

In some aspects, the plurality of channels 52 have a depth from 10 μm to 200 μm. Optionally, the plurality of channels 52 have a depth from 60 μm to 150 μm. In some aspects, the plurality of channels have a width from 50 μm to 500 μm. Optionally, the plurality of channels have a width from 150 μm to 300 μm. In some aspects, the plurality of channels can be V-shaped in cross sectional planes perpendicular to their dimension of elongation. In other aspects, the channels can be U-shaped, trapezoidal, rectangular, hemi-cylindrical, or any suitable shape.

In some aspects, the substrate 50 can comprise a sheet wound around the take-up roll 20 (forming one layer per rotation of the take-up roll). In other aspects, the substrate 50 can comprise a plurality of (e.g., a pair of) sheets that are simultaneously wound around the take-up roll 20. Accordingly, the system 10 can comprise a plurality of supply rolls 40 that simultaneously feed respective sheets to the take-up roll 20. The plurality of sheets can cooperate to form the substrate 50.

Optionally, the substrate 50, once wound onto the take-up roll 20, can comprise at least one region devoid of channels. In these aspects, the substrate can comprise at least one inner layer 60 that is devoid of, or substantially devoid of, channels. For example, the substrate 50 can comprise a plurality of layers without channels. In additional aspects, the substrate can comprise at least one outer layer 62 that is devoid of, or substantially devoid of, channels. In further aspects, the substrate can comprise one or more innermost layers and one or more outermost layers, wherein each of the innermost and outermost layers are devoid of channels. In this way, the innermost layers and outermost layers can serve to provide structure to the roll, by providing greater surface area between adjacent layers, thereby forming stronger bonds than the layers with channels. In some aspects, the substrate can have from about 3 to about 20 inner layers that are devoid of or substantially devoid of channels. In some aspects, the substrate can have from about 3 to about 30 outer layers that are devoid of or substantially devoid of channels. In some aspects, the substrate 50 can be stretched as it is wound around the take-up roll 20. For example, the substrate 50 can be stretched during an entirety of the rolling or during rolling of one or more outer layers that are configured to compress the substrate thereunder.

Accordingly, the take-up roll 20 can apply tension to the substrate to stretch the substrate is it is wound on the take-up roll.

According to additional exemplary aspects, the plurality of channels 52 can be formed into the substrate 50, and the supply roll 40 can include the substrate with the plurality of channels 52 formed therein. That is, the substrate can be provided on a pre-patterned roll (or a plurality of pre-patterned rolls), which can optionally comprise silicone sheeting. Accordingly, in these aspects, the system 10 can omit (be free of) the channel forming device.

In various aspects, the plurality of channels 52 can be formed into the substrate 50 prior to forming the supply roll 40. The plurality of channels 52 can be formed into the substrate 50 by one or more of: laser engraving, roll-to-roll embossing, molding (e.g., inline molding), or cutting (e.g., via a knife).

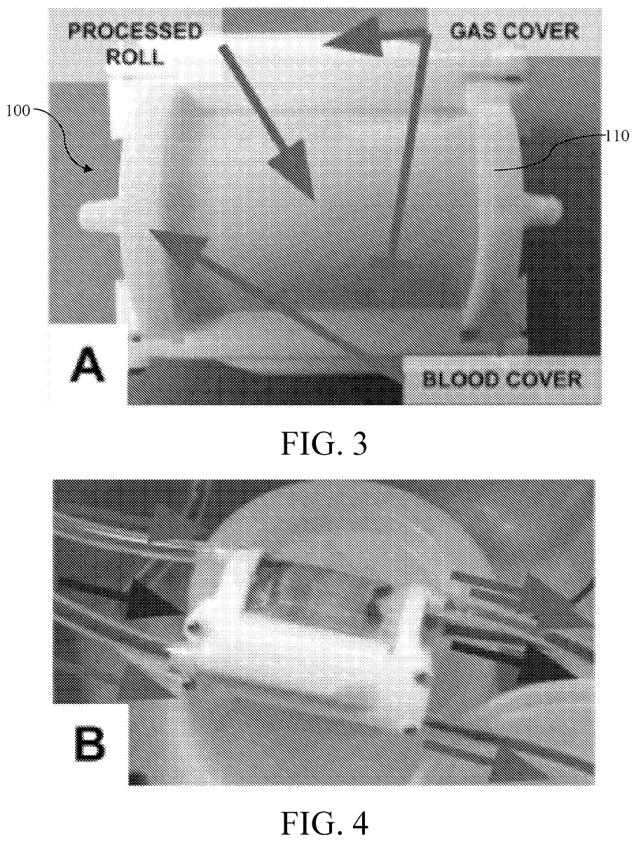

Referring to FIGS. 3, 6, and 7, a microfluidic lung 100 can comprise a substrate 50 wound into a roll, the substrate 50 having a plurality of channels 52 formed therein. The substrate 50 has a first surface 54 and a second surface 56 opposite the first surface. At least a portion of the first surface 54 of the substrate 50 is bonded to an adjacent portion of the second surface 56 of the substrate 50.

The microfluidic lung 100 can further comprise a housing 110. The substrate 50 wound into a roll can be positioned within the housing. The housing 110 can comprise at least one first fluid inlet and at least one first fluid outlet, wherein each of the at least one first fluid inlet and the at least one first fluid outlet is in communication with the first plurality of channels 52a. The housing 110 can comprise at least one second fluid inlet and at least one second fluid outlet, wherein each of the at least one second fluid inlet and the at least one second fluid outlet is in communication with the second plurality of channels 52b. The housing 110 can communicate a first fluid (e.g., gas) into and from the first plurality of channels and a second fluid (e.g., blood) into and from the second plurality of channels 52b without allowing the first and second fluids to mix.

Non-limiting examples of the disclosed devices, systems, and methods are provided in the following description.

### Example 1

### Overview

The ability to manufacture high-surface area microfluidic devices (e.g., PDMS devices with microstructures) can bring small-scale technologies from the realm of research to clinical and commercial applications. PDMS is a popular material used in implantable and microfluidic devices, known for its biocompatibility and even hemocompatibility. It is chemically inert, mechanically compliant, and optically translucent. Further, PDMS is gas permeable, allowing it to be used for membrane oxygenation.

In particular, research of a single-layer rolled microfluidic artificial lung (uAL) demonstrated that their clinical application would benefit from the ability to create devices with many layers of microfluidics. Disclosed herein is an automated manufacturing system, capable of manufacturing a cylindrical multi-layer prototype uAL.

### Methods

Using a combination of commercially available hardware, electronics, and custom-made components, an automated roll-to-roll manufacturing system (FIG. 1) was made. The system uses a rolled sheet of PDMS, as packaged by the manufacturer. A CO₂ laser engraver (Heavily-modified Omtech K40, with N₂ purge and the bottom cut out) creates arbitrary patterns in the PDMS sheet (FIG. 2). The sheet is then plasma-activated using two cold-plasma wands, which causes the PDMS to adhere to itself in an irreversible bond. Finally, it is rerolled into a processed device, around a 30 mm cast-PDMS core. Guide rods and sensors throughout the system ensure alignment.

This entire process is performed simultaneously and synchronously. Accordingly, only a fixed length of the PDMS needs to be unrolled at a time. This contrasts with conventional manufacturing methods in which the entire length which needs to be unraveled at once, placing practical limitations on the surface area of manufactured devices. This synchronization and automation is controlled at a system-level by an Arduino Mega 1680 and various supporting electronics. An Arduino UNO with CNC shield replaces the motherboard of the laser engraver, allowing synchronization between the laser engraver and the rest of the system.

### Results

Using the automated manufacturing system, a 27-layer cylindrical prototype μAL was manufactured, comprising 1) an 8-layer base 2) 11 layers of alternating blood and gas patterns 3) and an 8-layer protective cover. The prototype comprises 60 μm deep and 180 μm wide triangular channels (FIG. 2). This was placed into a custom housing with flowpaths (FIG. 3). Colored water was routed through the device for visualization (FIG. 4).

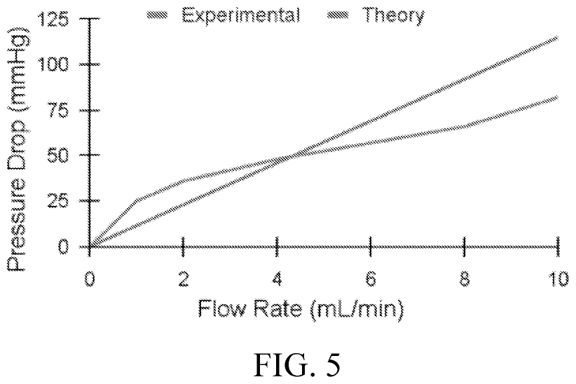

Empirical data pressure drop data was gathered for flows from 1 ml/min to 10 ml/min, which was in a similar range as theoretical values (FIG. 5).

If unrolled, the microstructure part of this system would stretch 1.1 m, and the total length would stretch 2.7 m. The system showed no signs of failing, and can accommodate many more layers.

### Conclusion

This roll-to-roll manufacturing system can be used to manufacture high-surface area cylindrical devices with microstructures that would be impractical to manufacture by conventional means. This can be employed for large-scale membrane gas exchanges such as human-scale μALs.

### Example 2

### Introduction

The ability to cost-effectively produce high-surface area microfluidic devices can bring many small-scale technologies from the realm of research to clinical and commercial applications. In particular microfluidic artificial lungs (uALs) (artificial lungs also known as membrane exchangers, membrane oxygenators, gas exchangers) promise improved gas exchange efficiency through micron-scale feature sizes. However, most uALs have only a single layer which limits their rated flows. Efforts to scale up these devices, such as by stacking multiple flat uALs have been labor intensive and resulted in bulky devices. Disclosed herein is an automated roll-to-roll (R2R) manufacturing system and a cylindrical multi-layer uAL.

### The Automated System:

The R2R system (FIG. 1) begins with a roll of biocompatible silicone rubber (Silpuran 2030). A heavily-modified CO₂ laser engraver (Omtech K40 with custom vacuum engraving bed, N₂ purge, and custom motherboard) creates patterns in the silicone sheet. The sheet is then plasma-activated using two CeraPlas F-series cold-plasma wands to bond the PDMS to itself in an irreversible bond. Finally, it is rerolled into a processed device around a 30 mm PDMS core. This entire process from unrolling to rerolling is performed simultaneously and synchronously, allowing the creation of very large area microfluidic devices. Further, it is automated (e.g., using an Arduino Mega 2560 controller), eliminating human error over this repetitive process.

### The Microfluidic Artificial Lung

In addition, disclosed herein is a μAL that can be manufactured using this system. The μAL begins with a silicone core (which can be plasma activated). There are several layers without fluidics meant to ensure the system enters a steady-state. This is followed by many alternating layers of blood and gas channels, oriented orthogonally such that the blood travels axially through the system, and the gas travels circumferentially. Gas is exchanged both inwards towards the center, and outwards away from the center. After the channels are engraved, the system is adjusted such that the PDMS is stretched as it wraps around the cylinder, compressing all the layers underneath it and ensuring solid adhesion, particularly where the engraved channels reduce the surface area available for plasma activation.

After the R2R machine is finished processing, the cylinder is cut with a scalpel to expose the microfluidic gas and blood channels. It is then installed into a custom 3D printed housing which routes blood and gas to their respective channels.

### Results

Using our automated manufacturing system, a 40-layer cylindrical prototype pAL was fabricated, comprising 1) an 10-layer base, 2) 15 layers of alternating blood and gas channels, and 3) an 15-layer cover for protection and compression. The thickness of the PDMS used in this design is 100 μm, and the laser engraved fluidic channels are triangular, approximately 60 μm deep and 180 μm wide. Water was pumped at 50 μl/min and 100 μl/min through half the lung, and CO₂ through the other. At 50 μl/min, water-side pressure drop was measured at 145 mmHg. At 100 μl/min, the lung sustained pressures up to 200 mmHg. Over 3 hours, the system reduced the pH of the water from 6.0 to 4.0, demonstrating functionality of the lung.

In exemplary aspects, it is contemplated that the exemplary devices, systems, and methods disclosed herein can be used in combination or conjunction with, or used to modify the devices, systems, and methods disclosed in U.S. patent application Ser. No. 16/499,999, which is incorporated herein by reference in its entirety.

### Example 3

Here, we report an automated manufacturing system capable of manufacturing a large area, cylindrical, multilayer PDMS microfluidic device, and use it to manufacture a series of uALs which were tested to ensure fluidic fidelity, pressure drop, and gas exchange.

### R2R System Overview and Design

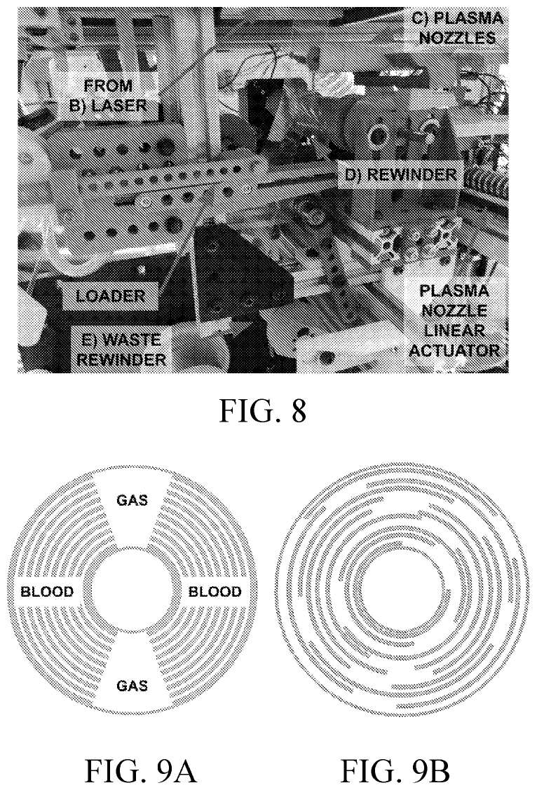

The R2R system (FIG. 1) begins with a roll of biocompatible PDMS rubber, which is unraveled to expose the sheet (also known as a “web” in R2R terminology). A CO2 laser engraver creates patterns in the PDMS sheet (FIG. 8). The sheet is then plasma activated to permit bonding the PDMS to itself in the next step. Finally, it is rerolled into a processed device around a PDMS core. This entire process from unrolling to rerolling is performed simultaneously and synchronously, removing processing space as a limitation to device size. The entire process is synchronized by an Arduino Mega. Most steps are automated to minimize human error over this repetitive process.

### Use of Radial Positioning

This R2R system is unique in that the final processed roll is used as a single device in its rolled-up state, and not unrolled again. The layer-to-layer radial position of the engravings can be fixed, so that the patterns on each layer line up radially, permitting access to the channels through vias (FIG. 9A). The length of PDMS engraved and used on each layer must increase to match the circumference of each successive layer. This differs from most other R2R systems, in which the processed roll must be unrolled again to access and/or separate the many individual small-scale devices. On these other R2R systems, the radial position of the pattern on the processed roll does not matter (FIG. 9B).

### Unwinder

The unwinder (Industrial Automation Specialists) uses a capstan friction system to passively (i.e. no motors or feedback systems) maintain a fixed tension as the PDMS is unwound and consumed.

The source PDMS used is 100 μm-thick Silpuran 2030 mounted on a 100 μm-thick polyethylene terephthalate glycol (PETG) backing (discussed in the next section). The PDMS source roll is cut into 60 mm-wide sections to match the width of all the guide rods and final rewinder in the R2R system. (A cutting rig is pictured in FIG. 23.)

### Laser Engraving

The laser engraver is a heavily modified Omtech K40. First, its motherboard has been removed and replaced with a custom programmed Arduino Uno for more precise control and synchronization with the Arduino Mega (which synchronizes all aspects of the R2R process). Second, the bottom of the laser engraver has been cut out, allowing continuous pass-through of the film for engraving. The engraving table has been replaced with an adjustable vacuum table. When turned on, it sucks the PDMS flat against it to hold it still for engraving. This permits a fixed focal length for the laser and ensures a uniform engraving depth. Several other adjustments including linear rail and hall-effect limit switches (for increased mechanical accuracy), and compressed air-assist and custom fan shroud for cooling and debris removal improve the overall engraving quality to be sufficient for this application. Finally, after each engraving, Scotch Magic tape is used to remove any excess engraving debris. The final step of removing the excess engraving debris is manual in this iteration of the system but can be automated in the future.

### Protective Backing and Synchronization

When purchased, the PDMS comes on a protective PETG backing. This backing must be removed from the PDMS before the PDMS is combined into the finished device. As such, it is pulled by its own PETG waste motor which operates in sync with the rewinder.

This backing, unlike the PDMS, is not elastic and so can be relied on to maintain a fixed length. Therefore, the waste motor controls the movement of the PDMS film through the system.

### Plasma Activation

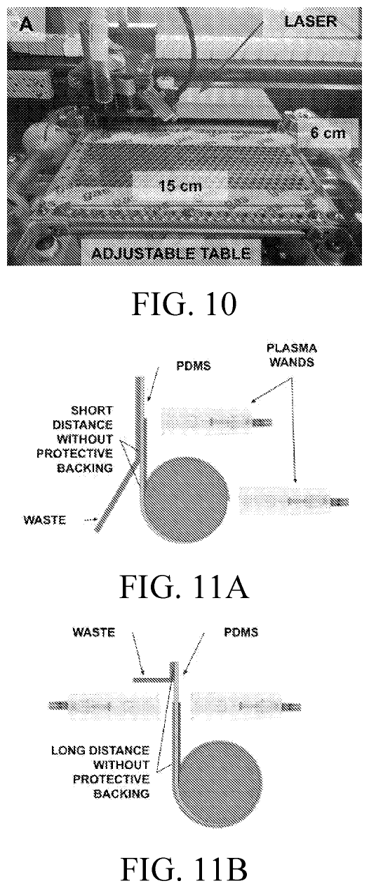

Both sides of the PDMS are plasma activated using two CeraPlas F-series cold-plasma wands (FIG. 10A). In a preliminary test, two neat sections of PDMS film were activated in this method then brought into contact where they bonded. When the two bonded PDMS films were then pulled apart, the individual sheets broke before the plasma-activated adhesion was undone.

Both wands are attached to a single linear actuator (RATTMMOTOR EBX1605-400 mm) which moves back and forth to ensure coverage across the entire width of the PDMS sheet. One plasma wand activates the engraved side of the PDMS while it is still attached to the PETG carrier. The other wand activates the back (i.e. non-engraved) side of the PDMS film after it has been integrated with the output roll on the rewinder (FIG. 11A). This configuration minimizes the distance between where the PETG backing is removed and where the PDMS film is bonded onto the output roll. If too long (see FIG. 11B for an example alternate configuration), the unbacked PDMS film can act unpredictably resulting in wrinkling and inconsistent tension. The alternate configuration shown in FIG. 11B would also require separate linear actuators for each plasma wand, increasing the overall size and complexity of the plasma mechanism.

### PDMS Tensioning

By using the PETG backing to control movement through the system, potential error due to PDMS stretching is minimized.

Since the progress of the PETG backing serves as a measure of how much PDMS is being consumed, adjusting how much the waste motor spins relative to the rewinder motor allows adjustment of the tension on the final roll/device. Normally, backing spins slightly slower than the rewinder, meaning that there is always a small amount of tension on the PDMS to avoid wrinkles when rerolling.

### Rewinder

The PDMS is then rolled into the rewinder, with the engraved side face-down, facing the center of the output roll. The rewinder is custom built around a NEMA-42 stepper motor. It clamps around a custom core assembly which can be easily installed for setup and removed once the machine has finished rolling the device. The rewinder rotates 360° to wrap a single layer of PDMS around each cycle. Since it spins exactly one circle each cycle, slight desynchronizations between where the patterns are engraved and where they end up on the finished core do not accumulate layer-to-layer.

### Device Layers and Engraving Phases

To begin the R2R process, a cylindrical substrate or core is placed on the (output) rewinder motor consisting of a two-layer structure. The internal layer is 3D printed ABS plastic for mechanical integrity. Fused (cured) to it is an outer layer of smooth, molded PDMS (MasterBond MasterSil 3231). This PDMS-coated core and beginning of the PDMS sheet are then plasma activated. After plasma activation, a spring-loaded roller pushes the beginning of the PDMS sheet onto the PDMS core to bond/fuse them. The roller is released after the machine runs for one core rotation, completing the process of bonding a single, unengraved layer of the PDMS film to the core.

Ten additional buffer layers of blank or non-engraved PDMS film allow the system to reach a steady state before the laser begins engraving the fluidic channels in subsequent layers. The laser engraves indexers (i.e. alignment markers) at the same position on each buffer layer. Based on the layer-to-layer error in the indexers on each buffer layer, the tension in the system is manually adjusted until there is negligible error in layer-to-layer alignment.

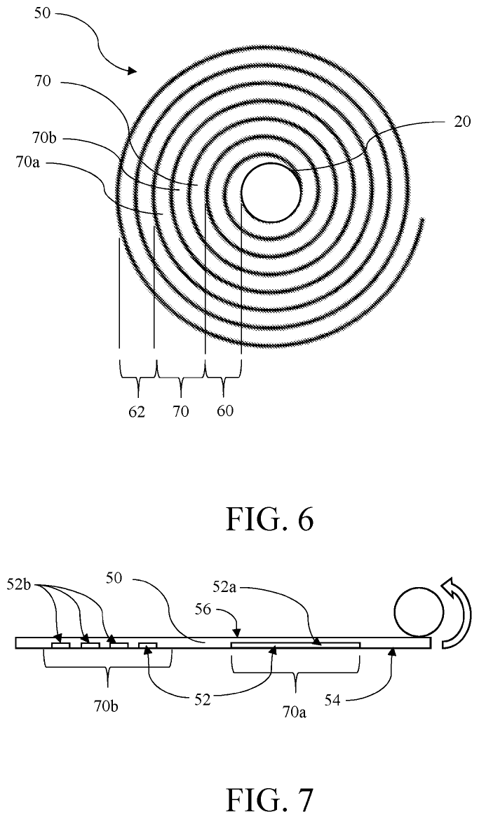

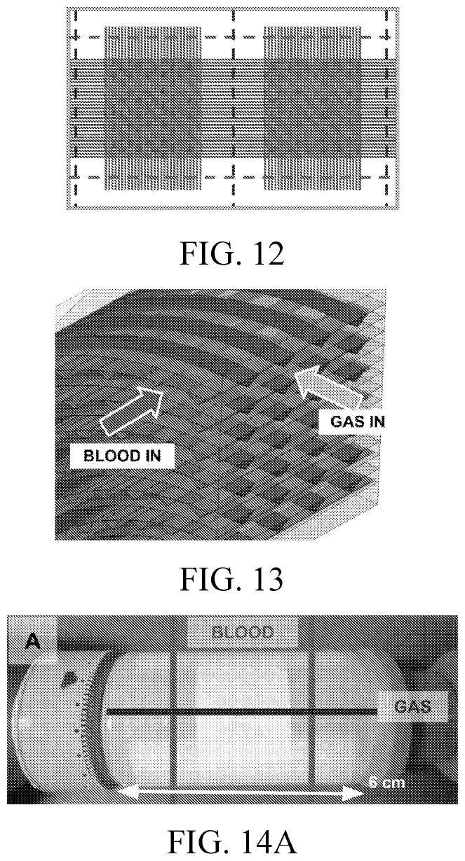

The laser engraving phase follows, alternating between gas and blood layers, with the engraving pattern length being equivalent to the current circumference of the rewinder output roll. Drawings of the gas and blood layers are shown in FIG. 12, where the blood (red) layers are engraved in one rotation, then the gas (blue) layers are engraved in the next rotation. These engravings result in orthogonally oriented blood and gas channels on alternating layers, as depicted in FIG. 13. The width of the drawing in FIG. 6 represents the current circumference of the output roll.

When all engraved layers are completed, fifteen non-engraved cover layers are added to the output roll to protect the engraved layers (e.g., from debris) prior to post processing (device manifolding and assembly). By intentionally slowing down the PETG rewinder, the PDMS is stretched as it is wrapped around the main rewinder. 24 mmHg of additional pressure resistance is added per layer. This compression of the final layers supplements the plasma adhesion in keeping the completed core secure.

### Device Assembly

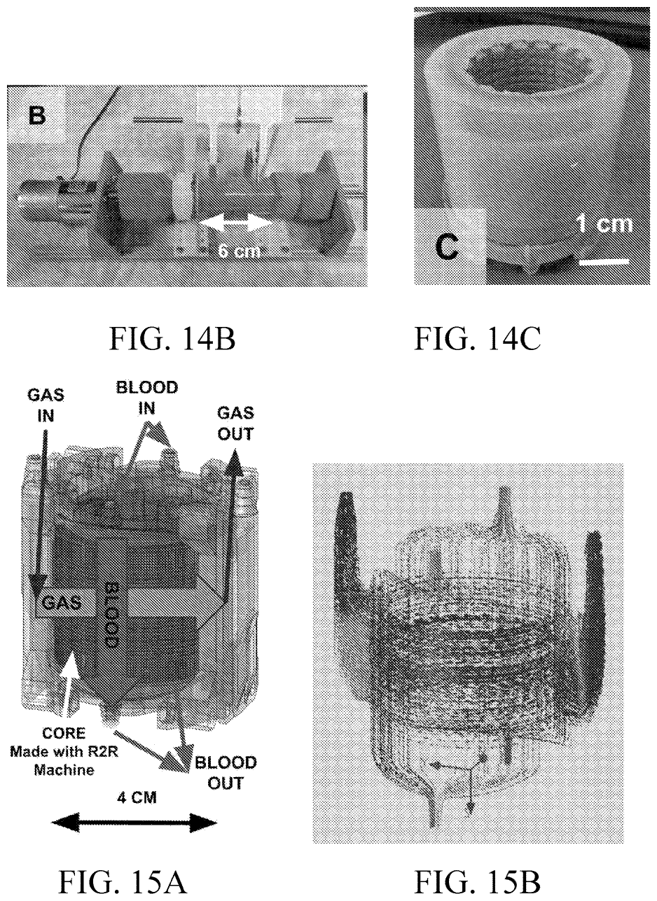

Upon completion of the R2R process, the core is removed from the machine. This completed core is cut to expose the blood channels using a specialized cutter (FIG. 14C). To assist this process, the internal layer of the core made of plastic is split into a middle main piece, and 2 side pieces. After the outer PDMS layer is cut to expose the blood layers, the two side pieces fall off and are discarded.

### Fluidic & Structural Housing

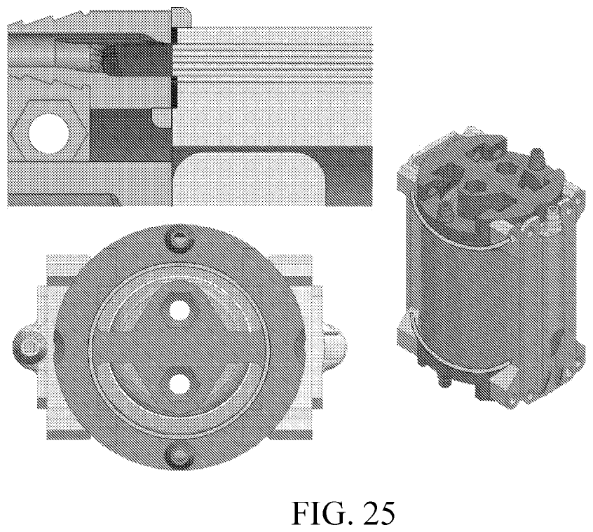

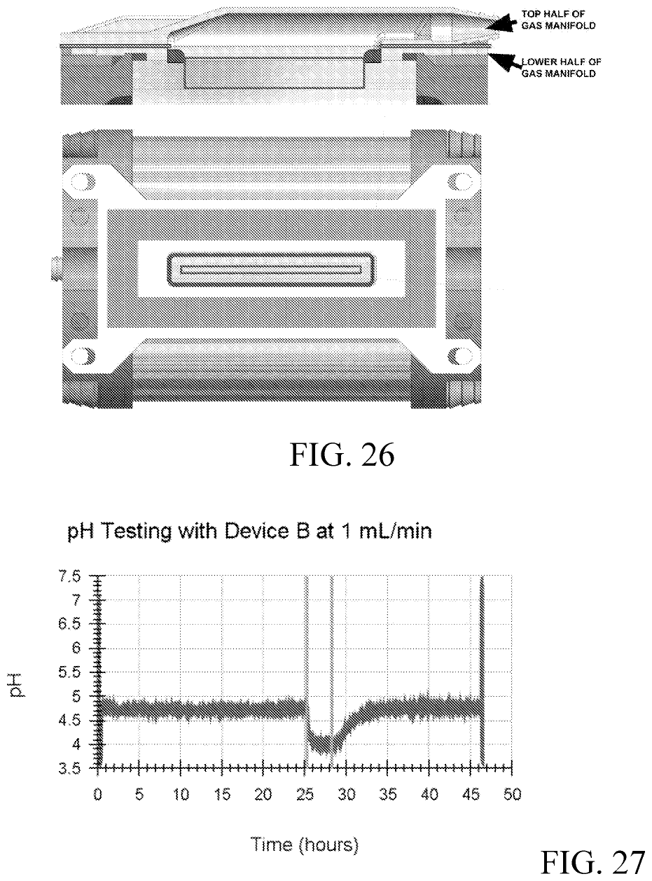

After cutting, the core is placed into a custom 3d-printed housing (Form 3 printer, RS-F2-GPCL-04 Clear Resin) sealed with silicone caulking. A more detailed housing model and caulking methodology is shown in FIG. 25. This housing serves to route gas and blood into their respective engraved flow channels. It also provides mechanical support for the core when it is cut along the horizontal lines in FIG. 14A to expose the gas channels. Note that there are two separate blood capillary sections, each comprising half the circumference of the rolled device, and with their own blood inlet and outlet.

### Leak Testing and Visualization Method

To visualize channel patency and check for leaks, each device was first filled with red (blood flowpath) and blue (gas flowpath) food coloring dissolved in water at a pressure of 100 mmHg (FIG. 17). This served to confirm that the device was properly constructed.

### Water Resistance Testing Method

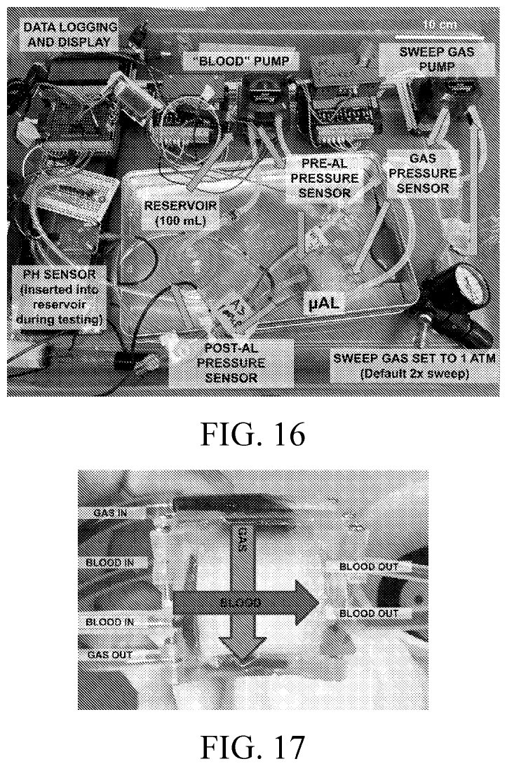

The in-vitro tests used a specialized μAL-testing setup shown in FIG. 16. In this circuit, water travels from a 100 mL reservoir through a peristaltic pump (Kamoer KPST-N14-C), through the blood side of the uAL, and back to the reservoir. Simultaneously, another peristaltic pump controls the sweep gas through the gas side of the uAL. Two pressure sensors (Panasonic ADP5140) before and after the uAL determine the blood-side pressure drop across the uAL, and a third pressure sensor before the uAL determines the gas-side pressure drop. A pH sensor (Atlas Scientific Gravity pH Sensor) in the reservoir can detect changes in CO2 concentration in the water.

To evaluate pressure drop, the blood-side flow rate of the system is gradually increased, while measuring the blood-side pressure drop. Each flow rate is maintained for 2 min, with the average pressure drop over the second minute being taken as the pressure drop at that flow rate. To avoid the risk of damaging devices before they reach the later tests, the pressure drop testing is terminated at approximately 200 mmHg.

### Water PH Testing Method

CO₂ was exchanged in water to qualitatively test the device's ability to perform gas exchange as a surrogate and precursor to blood testing. First, the water in the reservoir (FIG. 16) is conditioned by supplying compressed air (0% CO₂) through the gas side of the μAL and water through the blood CO₂. This removes any dissolved CO₂ present in the water. Then, the sweep gas through the μAL is changed to 100% CO₂ while the CO₂ content of the water reservoir is monitored via the pH sensor. Finally, the CO₂ is removed from the water by supplying compressed air to the gas side of the μAL again. A pre-calibration and post-calibration step^{‡} at the beginning and end of the test is performed using pH7 and pH4 buffer solutions. Sweep gas to water flow ratio was set to 0.5:1.0 to ensure that gas side pressure is less than water side pressure. Quantitative analysis cannot be performed^{‡} due to the inconsistency in water solutes, but the relative change in pH in a single run indicates successful CO₂ exchange.

### Blood Testing Method

Blood testing was performed following the FDA Cardiopulmonary Bypass Oxygenators 510(k) Submissions, published Nov. 13, 2000. This test determined the maximum flow rate at which blood entering the device at venous levels (65±5% O₂ saturation) would exit 95% saturated. To perform this test, the water in the reservoir was replaced with 250 mL of blood that was pre-conditioned (65±5% O₂ saturation, hemoglobin concentration 12±1 g/dL, pCO₂ 45±5 mmHg, base excess 0±5 mmol/L, temperature 37±2° C., and pH 7.4±0.1) and anticoagulated (active clotting time>1000 s).³⁰ A blood parameter monitoring system (Terumo CDI-500) was placed on the blood output of the μAL. This allowed monitoring of O₂ saturation. Blood flow varied between 0.4 and 4 mL/min with a sweep gas to blood flow ratio of 0.5:1.0 and outlet blood oxygenation was recorded. Each blood flow rate was allowed to settle over 1 minute, and the last 30 seconds was taken as the blood saturation value. The processed blood was then discarded and not returned to the reservoir.

### Results

### Manufacturing Results

Three devices were manufactured using the R2R system, each improving on the last. Design parameters are shown in Table 1.

Device A was the first successfully manufactured device. It contained a total of 1125 blood channels at a density of 10 channels/cm. The assembly procedure involved squeezing the silicone caulking between the housing and core.

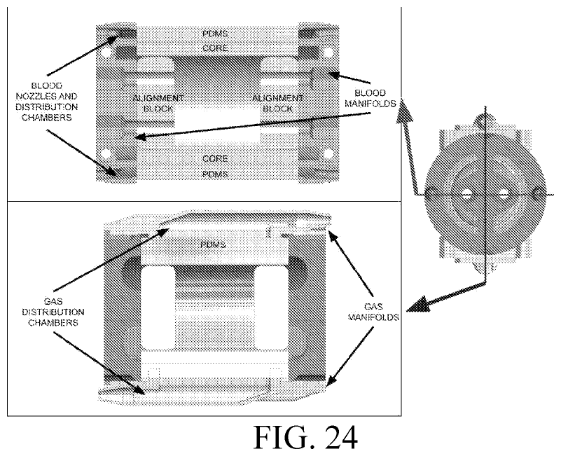

Device B improved upon Device A by containing a greater density of engraved channels. The device width, and thus the engraved blood channel length, was decreased to reduce device resistance and pressure drop, at the cost of a smaller increase in gas exchange surface area. Finally, it employed a newer housing and silicone caulking procedure in which the housing was squeezed against the core using bolts, and then sealed with silicone afterwards. (See FIGS. 24 and 25)

Device C built on the experience of previous runs, increasing the device layer count and engraved channel density.

TABLE 1
Properties of the three fabricated devices

| Device | A | B | C |
|---|---|---|---|
| Base Layer Count | 10 | 10 | 10 |
| Covering Layer Count | 15 | 15 | 15 |
| Theoretical Blood Layer Count | 7 | 7 | 12 |
| Gas Layer Count | 8 | 8 | 13 |
| Total Surface Area of Engraved Layers | 600 | 450 | 750 |
| (cm²) | | | |
| Total Length of Film (meters) | 3.0 | 3.0 | 4.0 |
| Length (cm) | 4.0 | 3.0 | 3.0 |
| Gas Spacing (mm) | 1.0 | 0.5 | 0.4 |
| Engravings per Gas Layer | 40 | 40 | 50 |
| Blood Spacing (mm) | 1.0 | 0.5 | 0.3 |
| Engravings per Blood Layer | 75 | 155 | 268 |
| Total Blood Engravings | 1125 | 2325 | 6700 |
| Theoretical Total Gas Exchange Surface | 10.75 | 28.49 | 106.1 |
| Area (cm²) | | | |

### Leak Testing and Visualization Results

For Device A, the food coloring failed to reveal flow in large sections of the device. Further, a leak was observed in one side of the blood flowpath and thus only half of the blood flowpath was functional for further testing. For Device B and Device C, no leaks were detected, and most observable channels were open.

### Water Resistance Testing Results

FIG. 17 shows the liquid side pressure drop and resistance for all devices. Device A exhibited a pressure drop of 200 mmHg at 0.4 mL/min. Higher flows were not tested. Device B exhibited a pressure drop of 167 mmHg at 4 mL/min. Device C exhibited a pressure drop of 223 mmHg at 4 mL/min. For all devices, the fluidic resistance (pressure/flow rate) decreased as flow rate increased.

To further validate device functionality, the ability to add O2 to blood was tested for Device B.

Pre-conditioned blood had a pH of 7.441, pCO₂ of 42 mmHg, pO2 of 37.3 mmHg, SaO₂ of 69.3%, and Hb of 12 g/dL (0.1608 mL of O₂/mL of blood) to match typical venous conditions. Outlet blood oxygen saturation vs blood flow rate plot for Device B is shown in FIG. 20, exhibiting the characteristic shape for blood oxygenators. At small flow rates, blood residence time in the device is large, permitting nearly full saturation of blood with O₂. As flow rate increases, residence time and blood oxygen saturation both decrease. FIG. 20 reveals a rated blood flow of 3.1 mL/min (outlet SO₂ of 95%) for this device.

### Analysis

### Manufacturing, Leak Testing, and Visualization Analysis

Device A demonstrated the milestone of having a multilayer R2R device with functional blood and gas channels in close proximity to each other, the prerequisites of micro-scale gas exchange. The device suffered from several shortcomings that were addressed in subsequent devices. First, based on visual inspection that food coloring was not seen in large sections of the lung, the amount of open blood capillaries was estimated to be equivalent to half of one blood layer. It is believed that this was due to the way the devices were sealed. When the silicone caulking was squeezed between the housing and the core, it spread laterally over the interface and occluded the channel openings. Secondly, the leak in one half of the device also exposed additional shortcomings in the quality of sealing. Finally, channel density was low (10 channels/cm) to maximize intermediate surface area for bonding. Although this increased the chance of success in this initial device, it resulted in both a lower number of capillaries and number of gas channels servicing the capillaries.

Device B and Device C both had successive improvements in the channel density and/or layer count compared to Device A. They additionally utilized a redesigned housing where the caulking was applied after the core was installed into the housing, such that the caulking was not squeezed over channel openings. Instead, 0.5+0.2 mm (equivalent to 5 blood layers) was intentionally occluded by the housing design to compensate for mechanical tolerance and misalignment.

Estimated device parameters taking into account the occluded channels are shown in Table 2.

TABLE 2
Properties of the three fabricated devices

| Device | A | B | C |
|---|---|---|---|
| Theoretical Blood Layer Count | 7 | 7 | 12 |
| Estimated Actual Blood Layer Count | 0.5 | 2 | 7 |
| (after caulking) | | | |
| Estimated Open Engraved Blood | 37.5 | 775 | 2680 |
| Channels | | | |
| Predicted Resistance | 367.8 | 13.35 | 3.86 |
| (mmHg/(mL/min)) | | | |

### Water Resistance Testing Analysis

All three devices exhibited pressure drops that increased with water flow rate, but which exhibited some unexpected properties.

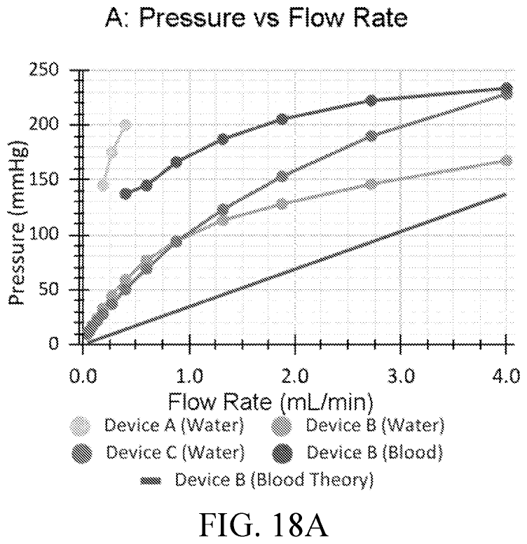

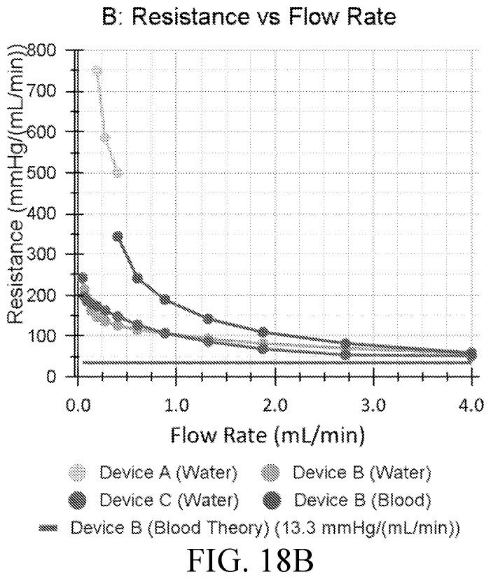

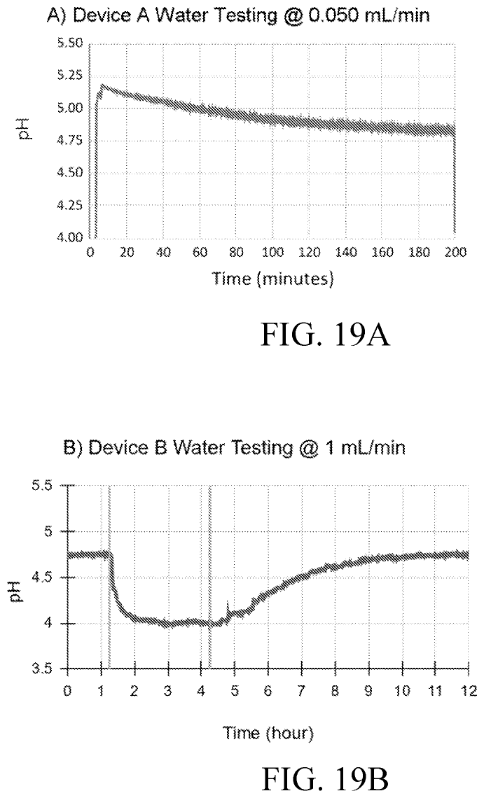

First, pressure drops were significantly higher than expected (FIGS. 18A-18B). Possible explanations for this include: A) the channels were much shallower than expected, B) debris within the channels greatly reduced their patency, and/or C) the channels were compressed by the pressure of the stretched covering layers. These explanations would have caused both smaller and/or fewer channels and higher pressure drops.

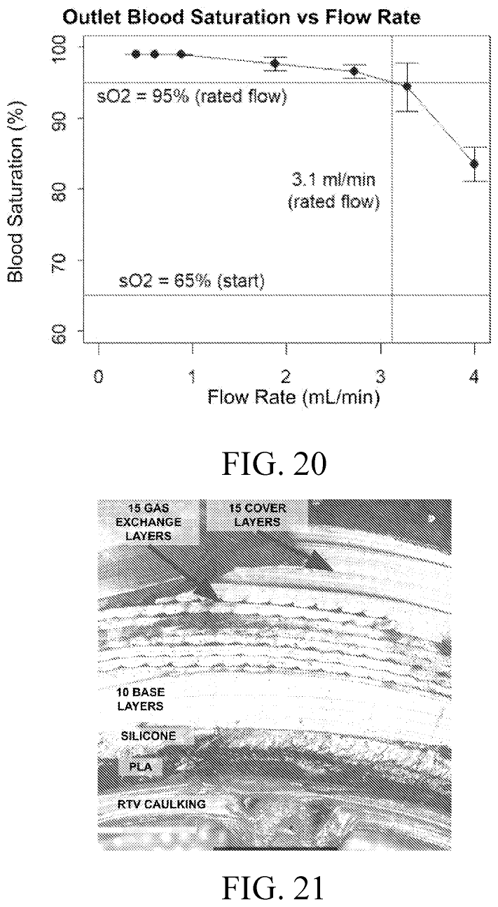

To investigate further, Device B was cut open with a scalpel to expose the channel cross section, and then imaged with a LEXT OLS4000 confocal microscope (FIG. 21). 95% confidence intervals of channel heights were 61±22 μm, channel widths were 221±73 μm, channel spacing was 520±57 μm, and layer height was 103±28 μm. Average values for channel widths and heights were roughly as expected. However, they had significantly more variation than expected.

One explanation for the increased resistance is that laser engraving depth was inconsistent along the length of the channels, causing some shallow regions. In this case, the resistance from the shallow areas would dominate. § Alternatively, debris in the channels could have reduced their patency.

Second, the rate of change of the pressure drop vs flow rate (i.e. the slope of the curve or fluidic resistance) of each device decreased with increasing pressure. This was unusual as pressure drop of microfluidic channels is typically proportional to flow rate (i.e. fluidic resistance is independent of flow rate). 11 One likely explanation is that pressure from the liquid dilated the flow channels, reducing channel resistance as the flow rate increased.

Finally, Device C exhibited similar (water side) fluidic resistance to that of Device B despite having many more capillaries. After manufacturing Device C, the laser was inspected. It was determined that the laser's cooling pump had stopped, likely causing a gradual deterioration in laser power and thus engraved channel depth over time. After this revelation, the R2R system was decommissioned for repairs and upgrades.

### Water pH Testing Analysis

Both Device A and Device B demonstrate the ability to change pH in water through CO₂ exchange. This indicates that both devices bring liquid and gas in close enough contact over a significant enough surface area to achieve gas exchange across the PDMS membrane.

### Blood Testing Analysis

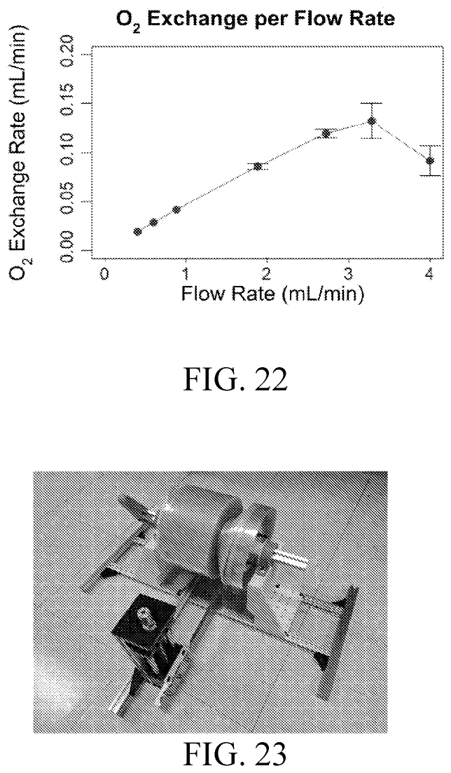

Oxygen exchange per flow rate is shown in FIG. 22. Total O₂ exchange by flow rate was calculated^{11,29} using saturation values from FIG. 20. 100% saturation was estimated to correspond with 0.1608 mL of O₂/mL of blood based on the hemoglobin content of 12 g/dL and 1.34 mL/O₂ per g/dL of hemoglobin.³⁰ Maximum O₂ exchange of 0.14 mL/min occurred at 3.3 mL/min blood flow.

Below its rated flow, the O₂ exchange rate of a typical AL increases linearly with increased flow. This is because the flow rate is increasing while outlet O₂ saturation is fixed at ~100%. Starting from about the rated flow, the O₂ saturation begins to drop from 100%. This causes the O₂ exchange rate to deviate from this linear trend. Then it reaches an asymptote as each increase in blood flow is counteracted by a proportional decrease in outlet blood SO₂. Eventually, it reaches an asymptote with maximum oxygen exchange that does not increase with blood flow rate.

For Device B, below 3.3 mL/min, the O₂ exchange rate seems to go up linearly as with other ALs. However, instead of gradually tapering into an asymptote, the O₂ exchange rate drops sharply at 4.0 mL/min.

According to FDA guidelines, the device has a rated flow of 3.1 mL/min. However, the total O₂ exchange decreases as flow increases between 3.3 mL/min and 4.0 mL/min (and presumably continues beyond 4.0 mL/min), which is unusual for oxygenators.¹¹ A possible explanation is that the increase in pressure dilates the channels, increasing the distance that O₂ must diffuse across blood to reach unbound hemoglobin molecules.

### Discussion

Overall, this work demonstrates a promising approach for the automated creation of large area microfluidic devices. A custom roll-to-roll manufacturing system was designed, assembled, and programmed to construct functional, many layer microfluidic artificial lungs. Detailed discussions of various aspects of the system are provided below.

Comparison with Current R2R Micromolding Technology

Soft lithography, or micromolding, has recently been applied to R2R systems to simultaneously create numerous single PDMS devices with micron-scale features.⁴ This approach makes use of a heated mold on a roller to imprint and cure PDMS as it travels through the R2R system. Curing times are typically <1 min for each imprint. Compared to the presented system, these micromold R2R systems offer accurate and smooth micron-scale features. However, these micromold R2R systems are only well suited for the creation of many, identical, small surface area PDMS devices. That is, the pattern on the imprinting roll in these systems is fixed. Without being able to change the diameter of the imprinting roll (and with it the length of the pattern) or the positioning of the patterns on the imprinting roll, there is no way to ensure the patterns consistently line up layer-to-layer to manufacture a single, large area device. The rolled nature of the processed fluidics is primarily a space-efficient storage method.

By contrast, the presented laser engraving based R2R method can create patterns that vary layer to layer and can be designed to match the current circumference of the output roll. This allows overlapping layers to align to each other, and by extension, the multi-layer fluidics to be accessed without unwrapping the processed output roll. These unique properties result in the first R2R method capable of manufacturing large area microfluidic devices.

The drawbacks of this laser based R2R process include: 1) relatively rough engraved patterns (compared to soft lithography/imprinting), 2) debris from laser engraving that is deposited on the PDMS surface, and 3) limitations in the engraved channel cross-sections (primarily triangular at the power levels and spot sized used here). Solutions to drawbacks 1 and 2 are the focus of ongoing studies and drawback 3 might be fixed by actively changing laser power, focal depth, and spot size.

### Insufficiency of Housing Caulking Method

The 0.5 mm of channels that was covered by overlap with the housing represents, amortized, the equivalent of 5 blood layers. This is a significant portion of the channels for the devices presented, which have 15-25 layers; however, this is expected to become increasingly negligible as the layer count reaches higher orders of magnitude. Further, because the device is manufactured using an automated system, adding 10 additional layers to compensate for this does not represent a significant effort.

### Insufficiency of Laser Engraving Cleaning

After each laser engraving step, there is significant debris on the PDMS surface despite the use of an air assist and a custom exhaust fan. The use of Scotch Magic Tape to remove debris from PDMS removes much, but not all this debris.

While the plasma adhesion forms an extremely strong bond when there are no engravings (the PDMS sheet will break before the plasma bond is undone), areas with gas or blood engravings had residual debris that decreased the bond strength resulting in a non-uniform and generally weak layer-to-layer bond. To compensate for this, the final PDMS capping layers (i.e. without engravings) were tensioned so that the tension from the PDMS assisted in preventing the active engraved layers from separating when the ends were cut. A more thorough removal of debris on engraved layers would ensure a more effective plasma activation and increased layer-to-layer bond strength while reducing/eliminating the need for layer tensioning.

### Mechanically Compliant Channels

The reduced resistance at higher pressures and flow rates is evidence that the channels expand when fluid is flowing through them (FIG. 18B). Although unusual, this may be beneficial for biocompatibility. Typically, low flows are associated with low shear and coagulation, and high flows with high shear and hemolysis.¹¹ However, if the channels expand as flow increases, the change in shear between different flow rates would reduce. Although PDMS μALs are known to be mechanically compliant,¹¹ to date, these are the only reported μALs which demonstrate the phenomenon to such an extent. This mechanical compliance would need to be investigated via a dedicated biocompatibility study in the future.

### Toward Full Automation

In the presented R2R system, only two processes require manual labor: 1) adjustment of the tensioning of the PETG carrier film and the PDMS film, and 2) the use of Scotch Magic Tape to remove laser engraving debris from the PDMS surface.

For 1), this intervention is only required during the base and covering layers, which is not expected to increase when scaling up to higher flow rates.

For 2), the manual removal of engraving debris would increase linearly when scaling up to larger surface area devices and would thus benefit from automation. It should be noted that this operation of removing debris is a relatively simple and risk-free process. During this research, there were no incidents of this process damaging a device. Therefore, although this operation prevents the machine from being left unattended, it does not significantly add to the risk of failure from human error.

### Toward Larger Area Microfluidic Devices

As this is the first iteration of the system and thus a proof of concept, the main goal of the work was to demonstrate an automated method to manufacture a multi-layer microfluidic device. This was achieved via the three prototype artificial lungs, culminating in Device C with 25 total active microfluidic layers. As currently designed, the R2R manufacturing system is likely only capable of making microfluidic devices with a maximum of 100 layers. Firstly, at ~100 layers, the diameter of the output roll would cause it to contact other components in the system including the plasma wands. Secondly, after approximately 100 layers, the engraved pattern for each layer would be larger than the surface area of the engraver table. Thus, to be able to create devices with more than 100 active layers, the system will need to be rebuilt and reprogrammed to both permit a larger output roll and to enable layers that are larger than the engraver table.

### More Complex Engraving Patterns

During this proof-of-concept run, engravings were limited to straight lines in the X and Y direction for simplicity. However, these engraved lines do not take full advantage of the abilities of the laser engraver, which can theoretically engrave diagonal lines, lines of varying depth, lines which intersect, and even rasterize a surface, thereby permitting the creation of microfluidic devices with many design topologies. A study to evaluate the feasibility of these features would reveal the full capability of this method.

### CONCLUSIONS

This manuscript presents a new, semi-automated roll-to-roll manufacturing method aimed at, for the first time, manufacturing large area microfluidic devices. The custom, integrated R2R system simultaneously and synchronically unrolls a PDMS film, engraves gas and blood channels into it, then rerolls and bonds the PDMS film into a cylindrical output roll. The resulting output roll, or core, is sealed into a custom housing to route gas and blood flowpaths. Three proof of concept μAL devices were manufactured to demonstrate the successful operation of the system. The μALs were tested with water and blood to verify flow patency and gas exchange. The best performing device had 15 active layers, for a total length of 3.0 m of PDMS and a total engraved surface area of 450 cm². This gave it a rated blood flow of 3.1 mL/min.

The following references are incorporated by reference herein in their respective entireties:
1 M. Smolka, S. Ruttloff, D. Nees, C. Prietl, V. Satzinger, B. Lamprecht, P. Hütter, J. Hesse, G. Kokkinis, G. Kriechhammer, D. Scheidl and B. Wilfing, Proceedings, 2018, 2, 1054.
2 K. Raj M and S. Chakraborty, Journal of Applied Polymer Science, 2020, 137, 48958.
3 Y. Xia and G. M. Whitesides, Angew Chem Int Ed Engl, 1998, 37, 550-575.
4 N. Bhattacharjee, A. Urrios, S. Kang and A. Folch, Lab Chip, 2016, 16, 1720-1742.
5 K. Jain, M. Klosner, M. Zemel and S. Raghunandan, Proceedings of the IEEE, 2005, 93, 1500-1510.
6 R. Liedert, L. K. Amundsen, A. Hokkanen, M. Mäki, A. Aittakorpi, M. Pakanen, J. R. Scherer, R. A. Mathies, M. Kurkinen, S. Uusitalo, L. Hakalahti, T. K. Nevanen, H. Siitari and H. Söderlund, Lab Chip, 2011, 12, 333-339.
7 R. Søndergaard, M. Hösel, D. Angmo, T. T. Larsen-Olsen and F. C. Krebs, Materials Today, 2012, 15, 36-49.
8 R. R. Søndergaard, M. Hösel and F. C. Krebs, Journal of Polymer Science Part B: Polymer Physics, 2013, 51, 16-34.
9 J. Hiltunen, C. Liedert, M. Hiltunen, O.-H. Huttunen, J. Hiitola-Keinänen, S. Aikio, M. Harjanne, M. Kurkinen, L. Hakalahti and L. P. Lee, Lab Chip, 2018, 18, 1552-1559.
10 A. L. Vig, T. Mäkelä, P. Majander, V. Lambertini, J. Ahopelto and A. Kristensen, J. Micromech. Microeng., 2011, 21, 035006.
11 J. A. Potkay, Lab Chip, 2014, 14, 4122-4138.
12 A. A. Gimbel, E. Flores, A. Koo, G. García-Cardeña and J. T. Borenstein, Lab Chip, 2016, 16, 3227-3234.
13 D. M. Hoganson, H. I. P. Ii, E. K. Bassett, I. D. Spool and J. P. Vacanti, Lab Chip, 2011, 11, 700-707.
14 B. C. Isenberg, E. M. Vedula, J. Santos, D. J. Lewis, T. R. Roberts, G. Harea, D. Sutherland, B. Landis, S. Blumenstiel, J. Urban, D. Lang, B. Teece, W. Lai, R. Keating, D. Chiang, A. I. Batchinsky and J. T. Borenstein, Adv Sci (Weinh), 2023, 10, e2207455.
15 T. Kniazeva, A. A. Epshteyn, J. C. Hsiao, E. S. Kim, V. B. Kolachalama, J. L. Charest and J. T. Borenstein, Lab Chip, 2012, 12, 1686-1695.
16 T. Kniazeva, J. C. Hsiao, J. L. Charest and J. T. Borenstein, Biomed Microdevices, 2011, 13, 315-323.
17 K. M. Kovach, M. A. LaBarbera, M. C. Moyer, B. L. Cmolik, E. van Lunteren, A. S. Gupta, J. R. Capadona and J. A. Potkay, Lab Chip, 2015, 15, 1366-1375.
18 J. A. Potkay, M. Magnetta, A. Vinson and B. Cmolik, Lab Chip, 2011, 11, 2901-2909.
19 J. A. Santos, A. A. Gimbel, A. Peppas, J. G. Truslow, D. A. Lang, S. Sukavaneshvar, D. Solt, T. J. Mulhern, A. Markoski, E. S. Kim, J. C.-M. Hsiao, D. J. Lewis, D. I. Harjes, C. DiBiasio, J. L. Charest and J. T. Borenstein, Lab Chip, 2021, 21, 4637-4651.
20 E. M. Vedula, B. C. Isenberg, J. Santos, W. Lai, D. J. Lewis, D. Sutherland, T. R. Roberts, G. T. Harea, C. Wells, B. Teece, J. Urban, T. Risoleo, D. Solt, S. Leazer, K. Chung, S. Sukavaneshvar, A. I. Batchinsky and J. T. Borenstein, ASAIO Journal, 2022, 68, 1312.
21 M. Dabaghi, G. Fusch, N. Saraei, N. Rochow, J. L. Brash, C. Fusch and P. Ravi Selvaganapathy, Biomicrofluidics, 2018, 12, 044101.
22 A. A. Gimbel, J. C. Hsiao, E. S. Kim, D. J. Lewis, T. F. Risoleo, J. N. Urban and J. T. Borenstein, Artif Organs, 2021, 45, E247-E264.
23 H. Matharoo, M. Dabaghi, N. Rochow, G. Fusch, N. Saraei, M. Tauhiduzzaman, S. Veldhuis, J. Brash, C. Fusch and P. R. Selvaganapathy, Biomicrofluidics, 2018, 12, 014107.
24 T. Rieper, C. Müller and H. Reinecke, Biomed Microdevices, 2015, 17, 86.
25N. Rochow, A. Manan, W.-I. Wu, G. Fusch, S. Monkman, J. Leung, E. Chan, D. Nagpal, D. Predescu, J. Brash, P. R. Selvaganapathy and C. Fusch, Artif Organs, 2014, 38, 856-866.
26 C. M. Leung, P. de Haan, K. Ronaldson-Bouchard, G.-A. Kim, J. Ko, H. S. Rho, Z. Chen, P. Habibovic, N. L. Jeon, S. Takayama, M. L. Shuler, G. Vunjak-Novakovic, O. Frey, E. Verpoorte and Y.-C. Toh, Nat Rev Methods Primers, 2022, 2, 1-29.
27 M. Yang, N. Sun, Y. Luo, X. Lai, P. Li and Z. Zhang, Biomicrofluidics, 2022, 16, 031503.
28 A. J. Thompson, L. H. Marks, M. J. Goudie, A. Rojas-Pena, H. Handa and J. A. Potkay, Biomicrofluidics, 2017, 11, 024113.
29 K. M. Kovach, J. R. Capadona, A. S. Gupta and J. A. Potkay, J Biomed Mater Res A, 2014, 102, 4195-4205.
30 C. for D. and R. Health, FDA.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising:
a laser engraver configured to form a plurality of channels into a substrate, the substrate having a first surface and a second surface opposite the first surface;
a take-up roll configured to roll the substrate with the plurality of channels formed therein; and
a bonder that is configured to bond a portion of the first surface of the substrate to an adjacent portion of the second surface of the substrate.

Aspect 2: The system of aspect 1, wherein the bonder comprises at least one cold-plasma wand.

Aspect 3: The system of aspect 2, wherein the at least one cold-plasma wand comprises a pair of cold-plasma wands spaced along a rotational axis of the take-up roll.

Aspect 4: The system of aspect 1, wherein the bonder comprises an oxygen plasma source, an ozone source for exposing the substrate to ozone, a device for providing corona discharge treatment, or an acid source for providing acid exposure.

Aspect 5: The system of any one of the preceding aspects, further comprising the substrate, wherein the substrate is provided on a supply roll.

Aspect 6: The system of aspect 5, wherein the supply roll further comprises a backing layer coupled to the second surface of the substrate.

Aspect 7: The system of aspect 6, further comprising a second take-up roll configured to wind the backing layer therearound to draw the backing layer from the substrate.

Aspect 8: The system of aspect 5, wherein the substrate comprises silicone.

Aspect 9: The system of aspect 8, wherein the substrate comprises PDMS.

Aspect 10: A method of making a microfluidic gas exchanger, the method comprising:
forming a plurality of channels into a substrate;
winding the substrate onto a take-up roll; and
bonding a first surface of a first portion of the substrate to a second surface of a second portion of the substrate that is adjacent the first portion along a radial axis.

Aspect 11: The method of aspect 10, wherein bonding the first surface of the first portion of the substrate to the second surface of the second portion of the substrate comprises using cold-plasma to bond the first surface of the first portion of the substrate to the second surface of the second portion of the substrate.

Aspect 12: The method of aspect 11, wherein using cold-plasma to bond the first surface of the first portion of the substrate to the second surface of the second portion of the substrate comprises using a pair of cold-plasma wands spaced along a rotational axis of the take-up roll.

Aspect 13: The method of any one of aspects 10-12, wherein the substrate is provided on a supply roll.

Aspect 14: The method of aspect 13, wherein the supply roll further comprises a backing layer coupled to the second surface of the substrate.

Aspect 15: The method of aspect 14, further comprising wind the backing layer around a second take-up roll to draw the backing layer from the substrate.

Aspect 16: The method of any one of aspects 10-15, wherein the substrate comprises silicone.

Aspect 17: The method of aspect 16, wherein the substrate comprises PDMS.

Aspect 18: The method of any one of aspects 10-17, wherein winding the substrate into the take-up roll comprises applying tension to the substrate to stretch the substrate is it is wound on the take-up roll

Aspect 19: The method of any one of aspects 10-18, wherein forming the plurality of channels into the substrate comprises engraving the plurality of channels into the substrate.

Aspect 20: The method of any one of aspects 10-19, wherein forming the plurality of channels into the substrate comprises one of: cutting the plurality of channels into the substrate, molding the plurality of channels into the substrate, or embossing the plurality of channels into the substrate.

Aspect 21: The method of any one of aspects 10-20, wherein winding the substrate onto the take-up roll comprises simultaneously winding a plurality of sheets from respective supply rolls onto the take-up roll.

Aspect 22: A system comprising:
a supply roll comprising a substrate, wherein a plurality of channels are formed into the substrate on the supply roll;
a take-up roll configured to roll the substrate with the plurality of channels formed therein; and
a bonder that is configured to bond a portion of the first surface of the substrate to an adjacent portion of the second surface of the substrate.

Aspect 23: A method of making a microfluidic gas exchanger, the method comprising:
winding a substrate onto a take-up roll, wherein the substrate comprises a plurality of channels formed therein; and
bonding a first surface of a first portion of the substrate to a second surface of a second portion of the substrate that is adjacent the first portion along a radial axis.

Aspect 24: The method of aspect 23, further comprising supplying the substrate comprising the plurality of channels formed therein from a supply roll.

Aspect 25: A microfluidic lung comprising:
a substrate wound into a roll, wherein the substrate comprises a plurality of channels formed therein, wherein the substrate has a first surface and a second surface opposite the first surface, wherein at least a portion of the first surface of the substrate is bonded to an adjacent portion of the second surface of the substrate.

Aspect 26: The microfluidic lung of aspect 25, wherein the plurality of channels have a depth from 10 μm to 200 μm.

Aspect 27: The microfluidic lung of aspect 25, wherein the plurality of channels have a width from 50 μm to 500 μm.

Aspect 28: The microfluidic lung of aspect 25, further comprising a housing, wherein the substrate wound into a roll is positioned within the housing.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A system comprising:
a laser engraver configured to form a plurality of channels into a substrate, the substrate having a first surface and a second surface opposite the first surface;
a take-up roll configured to roll the substrate with the plurality of channels formed therein; and
a bonder that is configured to bond a portion of the first surface of the substrate to an adjacent portion of the second surface of the substrate.

2. The system of claim 1, wherein the bonder comprises at least one cold-plasma wand.

3. The system of claim 2, wherein the at least one cold-plasma wand comprises a pair of cold-plasma wands spaced along a rotational axis of the take-up roll.

4. The system of claim 1, wherein the bonder comprises an oxygen plasma source, an ozone source for exposing the substrate to ozone, a device for providing corona discharge treatment, or an acid source for providing acid exposure.

5. The system of claim 1, further comprising the substrate, wherein the substrate is provided on a supply roll.

6. The system of claim 5, wherein the supply roll further comprises a backing layer coupled to the second surface of the substrate.

7. The system of claim 6, further comprising a second take-up roll configured to wind the backing layer therearound to draw the backing layer from the substrate.

8. The system of claim 5, wherein the substrate comprises silicone.

9. The system of claim 8, wherein the substrate comprises PDMS.

10. A method of making a microfluidic gas exchanger, the method comprising:
forming a plurality of channels into a substrate;
winding the substrate onto a take-up roll; and
bonding a first surface of a first portion of the substrate to a second surface of a second portion of the substrate that is adjacent the first portion along a radial axis.

11. The method of claim 10, wherein bonding the first surface of the first portion of the substrate to the second surface of the second portion of the substrate comprises using cold-plasma to bond the first surface of the first portion of the substrate to the second surface of the second portion of the substrate.

12. The method of claim 11, wherein using cold-plasma to bond the first surface of the first portion of the substrate to the second surface of the second portion of the substrate comprises using a pair of cold-plasma wands spaced along a rotational axis of the take-up roll.

13. The method of claim 10, wherein the substrate is provided on a supply roll, and wherein the method further comprises, before winding the substrate onto the take-up roll, supplying the substrate comprising the plurality of channels formed therein from the supply roll.

14. The method of claim 13, wherein the supply roll further comprises a backing layer coupled to the second surface of the substrate.

15. The method of claim 14, further comprising winding the backing layer around a second take-up roll to draw the backing layer from the substrate.

16. The method of claim 10, wherein the substrate comprises silicone.

17. The method of claim 16, wherein the substrate comprises PDMS.

18. The method of claim 10, wherein winding the substrate into the take-up roll comprises applying tension to the substrate to stretch the substrate is it is wound on the take-up roll

19. The method of claim 10, wherein forming the plurality of channels into the substrate comprises engraving the plurality of channels into the substrate.

20. The method of claim 10, wherein forming the plurality of channels into the substrate comprises one of: cutting the plurality of channels into the substrate, molding the plurality of channels into the substrate, or embossing the plurality of channels into the substrate.

21. The method of claim 10, wherein winding the substrate onto the take-up roll comprises simultaneously winding a plurality of sheets from respective supply rolls onto the take-up roll.

22. (canceled)

23. (canceled)

24. (canceled)

25. A microfluidic lung comprising:
a substrate wound into a roll, wherein the substrate comprises a plurality of channels formed therein, wherein the substrate has a first surface and a second surface opposite the first surface, wherein at least a portion of the first surface of the substrate is bonded to an adjacent portion of the second surface of the substrate.

26. The microfluidic lung of claim 25, wherein the plurality of channels have a depth from 10 μm to 200 μm and a width from 50 μm to 500 μm.

27. (canceled)

28. The microfluidic lung of claim 25, further comprising a housing, wherein the substrate wound into a roll is positioned within the housing.
